(19) United States
(12) Patent Application Publication
(10) Pub. No.: US 2007/0122880 A1
(43) Pub. Date: May 31, 2007

(54) Vector for the inducible expression of gene sequences

(75) Inventors: Christel GUILLOUF, Paris (FR); Anne Spinglard, Nanterre (FR)

Correspondence Address: NIXON & VANDERHYE, PC, 901 NORTH GLEBE ROAD, 11TH FLOOR, ARLINGTON, VA, 22203, US

(73) Assignee: Institut Curie, Paris (FR)

(21) Appl. No.: 11/550,045

(22) Filed: Oct. 17, 2006

Related U.S. Application Data
(60) Provisional application No. 60/727,854, filed on Oct. 19, 2005.

(51) Int. Cl. C12P 21/06; C07H 21/04; C07K 14/705

(52) U.S. Cl. 435/69.1; 435/320.1; 435/325; 530/350; 536/23.5

(57) ABSTRACT

The invention relates to a nucleic acid construct that comprises a human EF-1α promoter operatively associated with the tetracycline repressor (TetR). The invention further provides cell lines that have been stably transfected with such construct, as well as methods of using said construct, for the inducible expression of a transgene or for the inducible silencing of a endogenous sequence in mammalian cells.

The present application claims benefit of U.S. Provisional Ser. No. 60/727,854, filed Oct. 19, 2005, the entire contents of which is hereby incorporated by reference.

The invention relates to a vector enabling the efficient inducible expression of gene sequences (encoding proteins or interfering RNA) in mammalian cells.

### TECHNICAL BACKGROUND

Investigators have used two principal and reciprocal means to assess the physiological role of a given gene product in mammalian cells: overexpression of the gene or cDNA encoding the protein of interest, a situation that usually exacerbates its effects, and loss of function by similarity with the mutations generated in classical genetic studies. Sequences to be overexpressed are usually inserted downstream from strong promoters that drive their unregulated expression in the cells of interest. Gene silencing using RNA interference has become in the recent years a more time- and cost-effective alternative to homologous recombination, and more rigorous as well as less artefact-prone than the use of dominant-negative mutants, as a means to achieve loss of function studies in mammalian cells (Meister, G., and T. Tuschl. 2004. Mechanisms of gene silencing by double-stranded RNA. Nature. 431:343-349). The most frequently used way to study the effects of gene overexpression or silencing consists in the transient transfection (or viral infection) of a cell population with the appropriate expression vector or SiRNA, and analysis of the consequences in the following days. However, this method carries several caveats: (i) only a portion of the cell population may be transfected, and each individual cell may be affected to a variable extent; hence the result is a heterogenous population which may render the analysis and interpretation of the results, difficult; (ii) since each experiment is a new transfection, it requires careful characterization of the expression level (overexpression or silencing) of the protein of interest so as to ensure reproductibility between experiments. Moreover, such transfections, especially when they are performed on a large scale for biochemical experiments are very costly. These problems have often been overcome by resorting to the generation of stably transfected cell lines permanently overexpressing, or being silenced, for the protein of interest. This ensures homogeneity of the biological material, and careful control of the level of expression in all cells of the population. However, the stable over- or under-expression of many proteins may be growth-inhibitory or toxic; in many cases it leads to a permanent phenotypic modification of the cells, sometimes accompanied by secondary compensatory changes, which prevents a rigorous assessment of the physiological effects of the studied protein.

The tightly regulated inducible expression of proteins or gene silencing in mammalian cell lines has thus emerged as the method of choice for the rigorous assessement of their physiological effects without most of the artefacts associated with transient transfections or permanent expression as detailed above. To this end, expression of the foreign gene sequences or SiRNAs must be tightly controlled, and this should be achieved by using an inducer (or repressor) that in itself has no effect on the cells used in the study. Tetracyclin (Tet), an antimicrobial agent that has no deleterious effect on mammalian cells, has been widely used to control the expression of genes driven by polymerase 11 promoters. One system uses a chimeric protein that consists in the transactivation domain of virion protein 16 (VP16) fused to the Tet repressor (TetR) (Gossen, M., and H. Bujard. 1992. Tight control of gene expression in mammalian cells by tetracycline-responsive promoters. Proc Natl Acad Sci USA. 89:5547-51). This TetR-VP16 chimera strongly enhances transcription from minimal promoters upon binding to its cognate motif, the Tet operator; binding of Tet to the TetR in the TetR-VP16 chimera inhibits binding of the repressor to its binding site, thereby shutting off transcription when cells are treated with tetracyclin. Another system makes use of the physiological function of the Tet repressor: the Tet operator is inserted between the promoter and the coding sequences of the gene of interest. Binding of the Tet repressor to the Tet operator results in a transcriptional block of the promoter (Yao et al., 1998. Tetracycline repressor, tetR, rather than the tetR-mammalian cell transcription factor fusion derivatives, regulates inducible gene expression in mammalian cells. Hum Gene Ther. 9:1939-50.). This latter system has recently been adapted to regulate the expression of sequences driven by RNA polymerase III promoters, such as short hairpin RNAs leading to the generation of SiRNAs in cells which act to silence the expression of endogenous genes (van de Wetering, M et al, 2003. Specific inhibition of gene expression using a stably integrated, inducible small-interfering-RNA vector. EMBO Rep. 4:609-15).

### SUMMARY OF THE INVENTION

The inventors modified the latter system in order to adapt it to the tightly controlled inducible expression of proteins, as well as SiRNAs, to cell lines for which the previously described system is inefficient. They generated a novel inducible expression system. They more particularly created a vector for the stable expression of the Tet repressor in murine hematopoietic cells and immortalized embryo fibroblasts. They further created a cell line derived from NIH 3T3 fibroblasts. An optimized vector was also generated to achieve gene silencing in murine cells via the inducible expression of siRNAs.

The invention therefore provides a nucleic acid construct that comprises a human EF-1α promoter operatively associated with the tetracycline repressor (TetR). The invention further provides methods of using said construct, for the inducible expression of a transgene or for the inducible silencing of an endogenous sequence in a mammalian cell.

### BRIEF DESCRIPTION OF THE DRAWINGS

FIG. 1: Map of pcDNA6/TR-EF-1α vector.

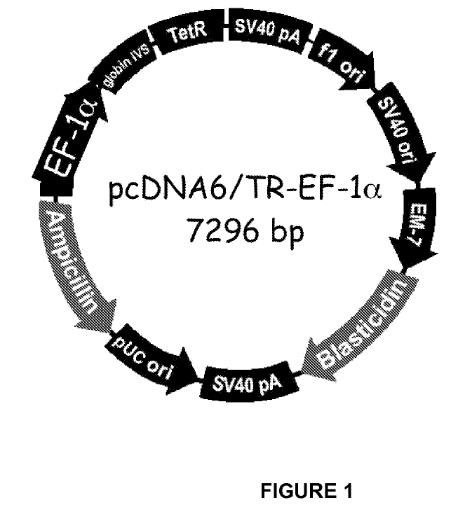

FIG. 2: Induction of luciferase expression by doxycyclin in NIH 3T3 Tet. NIH 3T3 and NIH 3T3 Tet cells were cotransfected with pcDNA4/TO-LucF and pRL-CMV-LucR. Renilla and firefly luciferase activities were measured in lysates of cells treated or not with 100 μg/ml doxycyclin for 24 h. The activity of firefly luciferase was normalized relative to the activity of the non-inducible renilla luciferase.

FIG. 3: Decrease of gem mRNA expression evaluated by quantitative real time RT-PCR by inducing siRNA against Gem mRNA. The histogram represents the decrease of Gem mRNA expression in presence of doxycycline relative to cell cultivated in absence of doxycycline, normalized to the Gapdh gene expression.

### DETAILED DESCRIPTION

The invention is based on use of the tetracyclin repressor to regulate the expression of genes controlled by the tetracyclin operator carried on a second plasmid, as described by (Yao et al., 1998 supra). The inventors have modified the system by driving expression of the tetracydin repressor from the human EF-1α promoter.

Polypeptide chain elongation factor 1α (EF-1α) is an eukaryotic counterpart of E.coli EF-Tu which promotes the GTP-dependent binding of an aminoacyl-tRNA to ribosomes. EF-1α is one of the most abundant proteins in eukaryotic cells, and expressed in almost all kinds of mammalian cells. The human chromosomal gene coding for EF-1α was isolated in 1990 and its promoter was shown to very efficiently stimulate the in vitro transcription (Uetsuki et al, 1989, J. Biol, Chem, 264:5791-5798; Mizushima, S., and S. Nagata. 1990. Nucleic Acids Res. 18:5322).

The inventors have now achieved highly inducible expression of target sequences in hematopoietic and fibroblast cell lines, exhibiting a highly regulated expression of foreign sequences by tetracyclin. The inventors have further inducibly expressed SiRNAs enabling tetracyclin-regulated gene silencing in murine hematopoietic and fibroblats cell lines.

### Definitions

The expression “nucleic acid construct” is understood to mean in particular a nucleic acid such as linear or circular DNA or RNA. It is also called a vector in the context of the present invention. It is most preferably a circle plasmid DNA vector.

The expression “selection sequence” is understood to mean a sequence which makes it possible to sort the cells which have integrated the nucleic acid construct of the invention and those in which the transfection has failed. A “positive” selection sequence refers to a gene encoding a product which allows only the cells carrying this gene to survive and/or to multiply under certain conditions. Among these “positive” selection sequences, there may be mentioned in particular the sequences of genes for resistance to an antibiotic, such as for example neomycin, ampicilin, hygromycin, puromycin, zeoycin, blasticidin or phleomycin. Another possible selection sequence is hypoxanthine phosphoribosyl transferase (HPRT). Cells which carry the HPRT gene can grow on HAT medium (containing aminopterin, hypoxanthine and thymidine), while the HPRT-negative cells die on the HAT medium.

The expression “transcription termination sequence” is understood to mean any sequence which makes it possible to stop the transcription, in particular a polyadenylation (polyA) site. It may be a virus-derived polyA, in particular the “Simian Virus 40” (SV40) polyA, or a polyA derived from a eukaryotic gene, in particular the polyA of the gene encoding Phosphoglycerate Kinase (pgk-1), or the polyA of the gene encoding rabbit .beta.globin.

“Tetracycline repressor” and “tetR” are used interchangeably herein to mean a sequence that encodes a polypeptide that exhibits both 1) specific binding to tetracycline and/or tetracycline derivatives; and 2) specific binding to tetO sequences when the tetR polypeptide is not bound by tetracycline or a tetracycline analog(s). “TetR” is meant to include a naturally-occurring (i.e., native) tetR sequence and functional derivatives thereof.

A “tetracycline operator” or “tetO” sequence can be obtained, for example, according to Hillen & Wissmann in Protein-Nucleic Acid Interaction, Topics in Molecular and Structural Biology, Saenger and Heinemann (eds.), Macmillan, London, Vol. 10, pp. 143-162 (1989), herein incorporated by reference with respect to the description and sequence of tetO. Other tetO sequences that can also be used in the practice of the invention. Several copies of the tet operator sequence can be used.

A “promoter” or “promoter sequence” is a DNA regulatory region capable of binding RNA polymerase in a cell and initiating transcription of a downstream (3′ direction) coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site (conveniently defined for example, by mapping with nuclease SI), as well as protein binding domains (consensus sequences) responsible for the binding of RNA polymerase. The promoter may be operatively associated with other expression control sequences, including enhancer and repressor sequences.

Promoters which may be used to control gene expression include, but are not limited to, cytomegalovirus (CMV) promoter (U.S. Pat. No. 5,385,839 and U.S. Pat. No. 5,168,062), the SV40 early promoter region (Benoist and Chambon, Nature 1981,290:304-310), the promoter contained in the 3′ long terminal repeat of Rous sarcoma virus (Yamamoto, et al., Cell 1980, 22:787-797), the herpes thymidine kinase promoter (Wagner et al., Proc. Natl. Acad. Sci. USA 1981, 78: 1441-1445), the regulatory sequences of the metallothionein gene (Brinster et al., Nature 1982, 296:39-42); prokaryotic expression vectors such as the beta-lactamase promoter (Villa-Komaroff, et al., Proc. Natl. Acad. Sci. USA 1978,75:3727-3731), or the tac promoter (DeBoer, et al., Proc. Natl. Acad. Sci. USA 1983, 80:21-25); see also “Useful proteins from recombinant bacteria” in Scientific American 1980, 242:74-94; promoter elements from yeast or other fungi such as the Gal 4 promoter, the ADC (alcohol dehydrogenase) promoter, PGK (phosphoglycerol kinase) promoter, alkaline phosphatase promoter; and transcriptional control regions that exhibit hematopoietic tissue specificity, in particular: beta-globin gene control region which is active in myeloid cells (Mogram et al., Nature 1985,315:338-340; Kollias et al., Ce II 1986, 46: 89-94), hematopoietic stem cell differentiation factor promoters, erythropoietin receptor promoter (Maouche et al., Blood 1991, 15:2557), etc.

A sequence is “under the control of” or “operatively associated with” transcriptional control sequences in a cell when RNA polymerase transcribes the sequence into RNA, particularly mRNA.

The term “transfection” means the introduction of a foreign nucleic acid into a cell.

By a “gene sequence of interest” is meant any nucleotide or DNA sequence that encodes a protein or interfering RNA or other molecule that is desirable for expression in a target cell (e.g., for production of the protein or other biological molecule (e.g., a therapeutic cellular product) in the target cell).

### Vectors

A first subject of the invention is thus a nucleic acid construct that comprises a tetracycline repressor (TetR) operatively associated with a EF-1α promoter, preferably a human EF-1α promoter.

The nucleic acid construct may more particularly comprise

(a) a tetracycline repressor (TetR), operatively associated with a EF-1α promoter, and a transcription termination sequence;

(b) at least one positive selection gene operatively associated with a promoter;

(c) at least one replication origin for replication in a prokaryotic cell, and optionally at least one replication origin for replication in a eukaryotic cell.

In a preferred embodiment the nucleic acid construct is a circle plasmid vector comprising, in the following order,
a human EF-1α promoter,
an intronic sequence, preferably rabbit beta-globin intron II,
that are upstream a tetracycline repressor (TetR),
the latter being followed by a transcription termination polyA sequence;
a replication origin in a prokaryotic cell,
a replication origin in a eukaryotic cell,
a promoter that drives the expression of
a blasticidin resistance gene as the positive selection gene,
followed by a transcription termination polyA sequence.

Preferably there is a spacer (e.g. an intronic sequence) between the EF-1α promoter and the TetR element.

In a particular preferred example, it has the sequence set forth in SEQ ID NO:1, and is called pcDNA6/TR-EF-1α. The position of each element in SEQ ID NO:1 is as follows:
promoter EF-1a: bases 254-1539
rabbit beta-globin intron II: bases 1662-2234
TetR gene: bases 2318-2938
SV40 early polyadenylation sequence: bases 2980-3111
f1 origin: bases 3531-3959
SV40 promoter and origin: bases 3531-4309
EM-7 promoter: bases 4349-4415
Blasticidin resistance gene: bases 4416-4814
SV40 early polyadenylation sequence: bases 4972-5102
pMB1 (pUC-derived) origin: bases 5485-6155
bla promoter: bases 7155-7259 (complementary strand)
ampicilin (bla) resistance gene: bases 6300-7160 (complementary strand)

Host Cells:

Another subject of the invention is a host cell that comprises the nucleic acid construct as defined above.

In a preferred embodiment it is a eukaryotic cell, preferably a mammal cell. It can be an adherent or non adherent cell, such as hematopoietic cells.

Such cells include CHO cells, HeLa cells, 293T (human kidney cells), NIH 3T3 cells, UT7, K562, HL60 and M1.

Inducible Expression of Gene Sequences:

The host cell or cell line that has been stably transfected with the vector as described above, is useful in various methods for expressing the gene sequence of interest or siRNAs.

In a first embodiment, the invention provides a method for inducibly expressing a gene sequence of interest in a cell, which method comprises providing a host cell, most preferably a mammalian cell line, that stably comprises the nucleic acid construct as defined above, and transfecting said recombinant cell with a nucleic acid construct carrying a gene sequence of interest that is operatively associated with a promoter under the control of a Tet operator, wherein addition of tetracycline or tetracycline analog induces expression of the gene sequence of interest in the cell.

In another embodiment, the invention provides a method for inducibly silencing an endogenous sequence of interest in a cell, which method comprises providing a host cell, most preferably a mammalian cell line, that stably comprises the nucleic acid construct as defined above, and transfecting said recombinant cell with a nucleic acid construct carrying DNA sequences leading to the synthesis of SiRNAs directed against said endogenous sequence of interest, wherein said DNA sequences are operatively associated with a promoter under the control of a Tet operator, wherein addition of tetracycline or tetracycline analog induces synthesis of said SiRNAs and silencing of said endogenous sequence of interest.

Tetracycline analogs can be any one of a number of compounds that are closely related to tetracycline and which bind to the tet repressor with a Ka of at least about 10⁶ M⁻¹. Preferably, the tetracycline analogs binds with an affinity of about 10⁹ M⁻¹ or greater, e.g., binds with an affinity of about 10¹¹ M⁻¹. Examples of such tetracycline analogs include, but are not limited to those disclosed by Hlavka and Boother, “The Tetracyclines,” IN: Handbook of Experimental Pharmacology 78, R. K. Blackwood et al. (eds.), Springer-Verlag, Berlin—N.Y., 1985; Mitschef, “The Chemistry of the Tetracycline Antibiotics,” Medicinal Research 9, Dekker, N.Y., 1978; Noyee Development Corporation, “Tetracycline Manufacturing Processes,” Chemical Process Reviews, Park Ridge, N.J., 2 volumes, 1969; Evans, “The Technology of the Tetracyclines,” Biochemical Reference Series 1, Quadrangle Press, New York, 1968; and Dowling, “Tetracycline,” Antibiotics Monographs no. 3, Medical Encyclopedia, New York, 1955; each of which are incorporated herein by reference with respect to tetracycline analogs.

Materials and Methods

Plasmid Construction

pcDNA6/TR-EF-1a was constructed by replacing the CMV promoter of pcDNA6/TR (Invitrogen) (excised with Nhe1 and Spe1; the cleaved vector was blunted by treatment with the Klenow fragment of DNA polymerase I) and replaced by the human EF-1α promoter excised from pEF-BOS, a kind gift of Dr. Nagata (Mizushima, S., and S. Nagata. 1990. supra. pEF-BOS, a powerful mammalian expression vector. Nucleic Acids Res. 18:5322), by cleavage with Hind III and Xba I (and blunting with Klenow).

pmTER was derived from pTER (van de Wetering et al., 2003 supra) by replacing the human H1 promoter by its murine orthologue. The murine H1 promoter was obtained by PCR amplification from the 10G6 plasmid kindly provided by Dr. de Murcia (Ame et al, J Biol Chem, 2001; 276:11092-9) using two primers (5′ TCTTCTTCGAACGCTCTTGAAGGACGACG 3′ and 5′ CTCTTAGATCTCTATCACTGATAGGGACACTA 3′) respectively containing BstB1 and BgIII restriction sites. The resulting fragment (200 bp) was inserted into the pTER plasmid, kindly provided by Dr. Clevers, from which the human H1 promoter had been excised with BstB1 and BgIII. The replaced region was sequenced to ensure integrity of the plasmid.

Sequences encoding SiRNAs for the Gem GTPase were flanked with BamHI and HindIII restriction sites, and cloned into pmTER linearized with BgIII and HindIII using standard cloning procedures.

The sequence of pmTER is set forth as SEQ ID NO:2.

Transfection and Generation of Cell Lines

NIH 3T3 cells (obtained from the ATCC) were cultured in DMEM with 10% donor calf serum and transfected with the pcDNA6/TR-EF-1a vector using a calcium phosphate co-precipitation procedure. They were selected for resistance to 10 μg/ml blasticidin (Invivogen) and individual colonies were expanded. In order to assess their ability to express the firefly luciferase upon induction with doxycyclin (a tetracyclin analog with a longer half-life), cells in a 35 mm dish were co-transfected with 20 ng pcDNA4/TO-LucF (a kind gift from Dr. Clevers) and 10 ng pRL-CMV-LucR using Lipofectamine (Invitrogen) according to the manufacturer's instructions. Cells were further cultured for 24 h in the absence or presence of 100 ng/ml doxycyclin (Sigma-Aldrich). Cells were lysed and assayed for firefly and renilla luciferase activities using the Dual Glo™ Luciferase Assay system (Promega) in a luminometer (Lumat LB 9507, EGSG BERTHOLD). The amount of renilla luciferase expressed from the pRL-CMV-LucR, a constitutive expression vector for this luciferase, enables to normalize the firefly luciferase activity of the various samples for transfection efficiency. The clone exhibiting the minimal amount of luciferase expressed in the absence of doxycyclin, and maximal level of luciferase activity expressed in the presence of doxycyclin was retained for further studies and named NIH 3T3 Tet.

These latter cells were transfected with plasmid pmTERGem by a calcium phosphate co-precipitation method as above and transfected cells were selected for their ability to grow in the presence of 100 μg/ml zeocin (Invivogen). Resistant colonies were expanded and treated for 24 h in the presence or absence of 100 ng/ml doxycyclin. Total RNA was prepared using RNABle® (Eurobio) and the levels of Gem mRNA were measured by quantitative real time RT-PCR (reverse transcription using M-MLV-RT (Invitrogen); PCR using SYBR® Green PCR Master Mix (Applied Biosystems) and an Applied Biosystems 7500 Real-Time PCR system.

Results and Discussion

In order to generate a system which would be amenable to the inducible expression of both proteins and SiRNAs of interest, we chose to adapt the system originally described by (Yao et al., 1998 supra) based on the use of the intact Tet repressor and its binding to the Tet operator. Achievement of a tightly regulated highly inducible expression system requires the efficient and stable expression of the Tet repressor in the cell lines of interest. The human cytomegalovirus (CMV) promoter used in the original system is unsuitable for use in cells of the hematopoietic lineage, as well as NIH 3T3 mouse embryo fibroblasts because the high levels of protein expression achieved in the first passages following transfection are lost with time. In order to overcome this problem, we chose to replace the CMV promoter in the pcDNA6/TR vector expressing the Tet repressor by the human EF-1α promoter that had previously been shown to efficiently drive the expression of foreign proteins in various cell lines (Mizushima and Nagata, 1990, supra).

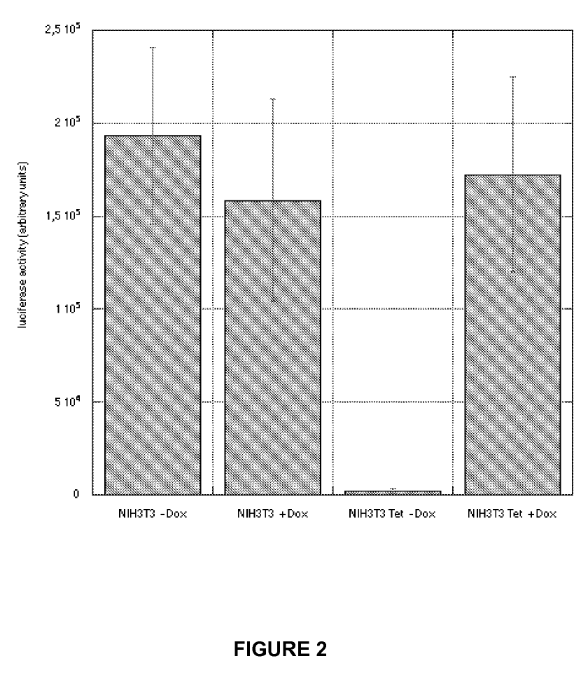

The resulting plasmid, pcDNA6/TR-EF-1α was then transfected into recipient mouse hematopoietic cell lines and NIH 3T3 cells, and colonies resistant to blasticidin were selected. Their capacity to enable the Tet-controlled expression of foreign sequences was assessed by transiently transfecting individual clones with a reporter vector, pcDNA4/TO-LucF driving the expression of firefly Luciferase from a CMV promoter under the control of a Tet operator. As shown in FIG. 2 for NIH 3T3 cells, we obtained a stable cell line, NIH 3T3 Tet, which in the absence of doxycyclin expressed a very low level of firefly Luciferase. In the presence of doxycyclin, Luciferase expression was induced to levels comparable with those achieved in parental NIH 3T3 cells devoid of Tet repressor. Under those conditions, we assessed that this cell line enabled to obtain the highly inducible expression of a foreign protein (induction by a factor of 90), a phenotype that was stable for at least 12 passages as well as freezing and thawing of the cell lines. Similar results were obtained in mouse hematopoietic cell lines (data not shown).

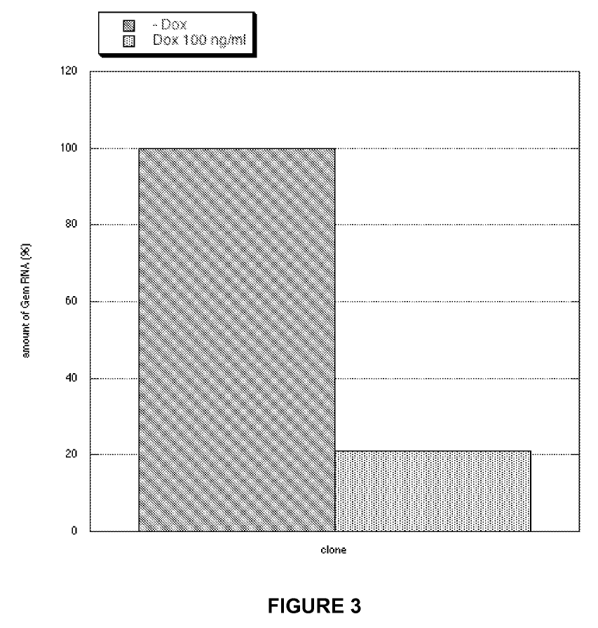

We then proceeded to use the NIH 3T3 Tet line to inducibly silence the gene encoding the Gem GTPase, a protein studied in our laboratory, in order to demonstrate the practical application of such a cell line. The inducible expression of SiRNAs, leading to inducible gene silencing, can be achieved by stably transfecting a cell line expressing the Tet repressor with a plasmid driving the expression of SiRNAs from an RNA polymerase III promoter containing a Tet operator sequence inserted between the promoter and the transcription start site, such as the pTER vector (van de Wetering et al., 2003 supra). In order to attain gene silencing in cell lines of murine origin with a maximal efficiency, we modified this latter vector by replacing the human H1 promoter by its mouse orthologue. DNA sequences leading to the synthesis of SiRNAs directed against the murine Gem protein were inserted, the resulting vector was transfected into NIH 3T3 Tet cells, and zeocin-resistant colonies were selected. They were individually tested for the expression of the Gem mRNA by RT-PCR after being treated for 24 h in the presence or absence of 100 ng/ml Doxycyclin, as described in the material and methods section. Up to 20% decrease of Gem mRNA expression compared to the untreated cells was obtained (FIG. 3).

Similar results were also obtained for the silencing of another gene in mouse hematopoietic cell lines (not shown).

### CONCLUSIONS

We have modified an existing inducible gene expression system in order to obtain with tight control and a high level of inducibility the expression of foreign proteins, as well as the silencing of endogenous genes, in murine hematopoietic cells and fibroblasts. Replacing the CMV promoter, originally used to drive the ectopic expression of the Tet repressor in cells, by the promoter of the human EF-1a gene was key to obtain a strong expression of the protein that was stable for many cell passages. This enabled to generate a derivative of the murine embryo fibroblast line NIH 3T3, NIH 3T3 Tet, stably expressing the Tet repressor, and therefore suitable for the inducible expression of foreign proteins, or inducible gene silencing through the inducible expression of SiRNAs. In this line, the expression level of a foreign protein such as luciferase in the presence of the inducer (doxycyclin) exhibited a high level of expression. This maximal expression level was similar to what is obtained in the parental NIH 3T3 line not expressing the Tet repressor, showing that the capacity of the cell line to overexpress a foreign sequence was not affected by the procedure leading to expression of the Tet repressor. In the absence of inducer, the residual level of expression of a foreign protein such as luciferase was slightly above background, but 90-fold lower than under fully induced conditions, showing a tight degree of expression control of the foreign gene. Importantly, we were unable to detect any decrease in the expression level of the Gem mRNA (using quantitative RT-PCR) in the absence of doxycyxlin.

The system that we have developed enables for the first time to generate mouse hematopoietic cell lines as well as derivatives from NIH 3T3 fibroblasts that stably exhibit a tight and highly inducible control of gene expression. This should prove to be a highly valuable tool to study the biology of hematopoietic cells. Moreover, NIH3T3 is a widely used cell line for the study of cell cycle and cellular responses to growth factors as well as to various signals that influence adherence, motility, cytoskeleton and gene expression. We therefore believe that the set of vectors that we have generated, pcDNA6/TR-EF-1a and pmTER, as well as the NIH 3T3 Tet line, are of great interest for biologists tackling the challenging issues described above.

All references cited herein are incorporated in their entirety by reference.

What is claimed is:

1. A nucleic acid construct that comprises a tetracycline repressor (TetR) operatively associated with a EF-1α promoter, preferably a human EF-1α promoter.

2. The nucleic acid construct of claim 1, which comprises
(a) a tetracycline repressor (TetR), operatively associated with a EF-1α promoter, and a transcription termination sequence;
(b) at least one positive selection gene, operatively associated with a promoter;
(c) at least one replication origin for replication in a prokaryotic cell, and optionally at least one replication origin for replication in a eukaryotic cell.

3. The nucleic acid construct of claim 2, that is a circle plasmid vector comprising, in the following order,
a human EF-1α promoter
an intronic sequence
that are upstream a tetracycline repressor (TetR),
the latter being followed by a transcription termination polyA sequence;
a replication origin in a prokaryotic cell,
a replication origin in a eukaryotic cell,
a promoter that drives the expression of
a blasticidin resistance gene as the positive selection gene,
followed by a transcription termination polyA sequence.

4. The nucleic acid construct of claim 3, that has the sequence set forth in SEQ ID NO:1, the construct being called pcDNA6/TR-EF-1α.

5. A host cell that comprises the nucleic acid construct of claim 1.

6. The host cell of claim 5, which is a eukaryotic cell, preferably a mammal cell.

7. The host cell of claim 6, that is an adherent or non adherent cell line.

8. The host cell of claim 6, which is a NIH 3T3 cell.

9. A method for inducibly expressing a gene sequence of interest in a cell, which method comprises providing a host cell that stably comprises the nucleic acid construct of claim 1, and transfecting said cell with a nucleic acid construct carrying a gene sequence of interest that is operatively associated with a promoter under the control of a Tet operator, wherein addition of tetracycline or tetracycline analog induces expression of the gene sequence of interest in the cell.

10. The method of claim 9, wherein the cell is a mammalian cell line.

11. A method for inducibly silencing an endogenous sequence of interest in a cell, which method comprises providing a host cell that stably comprises the nucleic acid construct of claim 1, and transfecting said cell with a nucleic acid construct carrying DNA sequences leading to the synthesis of SiRNAs directed against said endogenous sequence of interest, wherein said DNA sequences are operatively associated with a promoter under the control of a Tet operator, wherein addition of tetracycline or tetracycline analog induces synthesis of said SiRNAs and silencing of said endogenous sequence of interest.

12. The method of claim 11, wherein the cell is a mammalian cell line.


SEQUENCE LISTING (4 sequences)

SEQ ID NO: 1 - DNA, 7296 bp, artificial sequence
  Description of Artificial Sequence plasmid pcDNA6/TR-EF1a
gacggatcgggagatctcccgatcccctatggtgcactctcagtacaatctgctctgatgccgcatagttaagccagtatctgctccctgcttgtgtgttggaggtcgctgagtagtgcgcgagcaaaatttaagctacaacaaggcaaggcttgaccgacaattgcatgaagaatctgcttagggttaggcgttttgcgctgcttcgcgatgtacgggccagatatacgcgttgacattgattattgactagagctttgcaaagatggataaagttttaaacagagaggaatctttgcagctaatggaccttctaggtcttgaaaggagtgggaattggctccggtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgaggaattctctagctagcgtttaaacttaagcttggtacccggggatcctctagggcctctgagctattccagaagtagtgaagaggcttttttggaggcctaggcttttgcaaaaagctccggatcgatcctgagaacttcagggtgagtttggggacccttgattgttctttctttttcgctattgtaaaattcatgttatatggagggggcaaagttttcagggtgttgtttagaatgggaagatgtcccttgtatcaccatggaccctcatgataattttgtttctttcactttctactctgttgacaaccattgtctcctcttattttcttttcattttctgtaactttttcgttaaactttagcttgcatttgtaacgaatttttaaattcacttttgtttatttgtcagattgtaagtactttctctaatcacttttttttcaaggcaatcagggtatattatattgtacttcagcacagttttagagaacaattgttataattaaatgataaggtagaatatttctgcatataaattctggctggcgtggaaatattcttattggtagaaacaactacatcctggtcatcatcctgcctttctctttatggttacaatgatatacactgtttgagatgaggataaaatactctgagtccaaaccgggcccctctgctaaccatgttcatgccttcttctttttcctacagctcctgggcaacgtgctggttattgtgctgtctcatcattttggcaaagaattgtaatacgactcactatagggcgaattgatatgtctagattagataaaagtaaagtgattaacagcgcattagagctgcttaatgaggtcggaatcgaaggtttaacaacccgtaaactcgcccagaagctaggtgtagagcagcctacattgtattggcatgtaaaaaataagcgggctttgctcgacgccttagccattgagatgttagataggcaccatactcacttttgccctttagaaggggaaagctggcaagattttttacgtaataacgctaaaagttttagatgtgctttactaagtcatcgcgatggagcaaaagtacatttaggtacacggcctacagaaaaacagtatgaaactctcgaaaatcaattagcctttttatgccaacaaggtttttcactagagaatgcattatatgcactcagcgctgtggggcattttactttaggttgcgtattggaagatcaagagcatcaagtcgctaaagaagaaagggaaacacctactactgatagtatgccgccattattacgacaagctatcgaattatttgatcaccaaggtgcagagccagccttcttattcggccttgaattgatcatatgcggattagaaaaacaacttaaatgtgaaagtgggtccgcgtacagcggatcccgggaattcagatcttattaaagcagaacttgtttattgcagcttataatggttacaaataaagcaatagcatcacaaatttcacaaataaagcatttttttcactgcattctagttgtggtttgtccaaactcatcaatgtatcttatcatgtctggtcgaatccatcacactggcggccgctcgagtctagagggcccgcggttcgaaggtaagcctatccctaaccctctcctcggtctcgattctacgcgtaccggtcatcatcaccatcaccattgagtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattaattctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagcctttgtctcaagaagaatccaccctcattgaaagagcaacggctacaatcaacagcatccccatctctgaagactacagcgtcgccagcgcagctctctctagcgacggccgcatcttcactggtgtcaatgtatatcattttactgggggaccttgtgcagaactcgtggtgctgggcactgctgctgctgcggcagctggcaacctgacttgtatcgtcgcgatcggaaatgagaacaggggcatcttgagcccctgcggacggtgccgacaggtgcttctcgatctgcatcctgggatcaaagccatagtgaaggacagtgatggacagccgacggcagttgggattcgtgaattgctgccctctggttatgtgtgggagggctaagcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 2 - DNA, 4320 bp, artificial sequence
  Description of Artificial Sequence plasmid pmTER
gacggatcgggagatcccccgggctgcaggaattcgaacgctcttgaaggacgacgtcatcatcccttgcccggatgcgcgggcttcttgtctggcacaggagcctgggatagagcacatgcaaattacgcgctgtgctttgtgggaaatcaccctaaacgtaaaatttattcctctttcgagccttatagtgtccctatcagtgatagagatctaagcttggtaccgagctcggatccactagtccagtgtggtggaattctgcagatatccagcacagtggcggccgctcgagtctagagggcccgtttaaacccgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctctagggggtatccccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggggcatccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttggggatttcggcctattggttaaaaaatgagctgatttaacaaaaatttaacgcgaattaattctgtggaatgtgtgtcagttagggtgtggaaagtccccaggctccccaggcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctctgcctctgagctattccagaagtagtgaggaggcttttttggaggcctaggcttttgcaaaaagctcccgggagcttgtatatccattttcggatctgatcagcacgtgttgacaattaatcatcggcatagtatatcggcatagtataatacgacaaggtgaggaactaaaccatggccaagttgaccagtgccgttccggtgctcaccgcgcgcgacgtcgccggagcggtcgagttctggaccgaccggctcgggttctcccgggacttcgtggaggacgacttcgccggtgtggtccgggacgacgtgaccctgttcatcagcgcggtccaggaccaggtggtgccggacaacaccctggcctgggtgtgggtgcgcggcctggacgagctgtacgccgagtggtcggaggtcgtgtccacgaacttccgggacgcctccgggccggccatgaccgagatcggcgagcagccgtgggggcgggagttcgccctgcgcgacccggccggcaactgcgtgcacttcgtggccgaggagcaggactgacacgtgctacgagatttcgattccaccgccgccttctatgaaaggttgggcttcggaatcgttttccgggacgccggctggatgatcctccagcgcggggatctcatgctggagttcttcgcccaccccaacttgtttattgcagcttataatggttacaaataaagcaatagcatcacaaatttcacaaataaagcatttttttcactgcattctagttgtggtttgtccaaactcatcaatgtatcttatcatgtctgtataccgtcgacctctagctagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtc

SEQ ID NO: 3 - DNA, 29 bp, artificial sequence
  Description of Artificial Sequence primer containing BstB1 site
tcttcttcgaacgctcttgaaggacgacg

SEQ ID NO: 4 - DNA, 32 bp, artificial sequence
  Description of Artificial Sequence primer containing BglII site
ctcttagatctctatcactgatagggacacta